(19) United States
(12) Patent Application Publication
(10) Pub. No.: US 2002/0032354 A1
(43) Pub. Date: Mar. 14, 2002

(54) Process for manufacturing magnesium alkoxide

(75) Inventors: Burkhard Standke, Loerrach (DE); Hartwig Rauleder, Rheinfelden (DE); Michael Horn, Rheinfelden (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/951,618

(22) Filed: Sep. 14, 2001

(30) Foreign Application Priority Data
Sep. 14, 2000 (DE) 10045356.2

(51) Int. Cl. C07C 29/70

(52) U.S. Cl. 568/840

(57) ABSTRACT

The present invention relates to a process for manufacturing magnesium alkoxide by conversion of magnesium with an alcohol, characterized in that magnesium is used in piece form. The invention also relates to the use of magnesium alkoxide obtained according to the present invention as a feedstock for chemical or pharmaceutical processes, or for manufacturing catalysts or as an anhydrous neutralizing agent.

### BACKGROUND OF THE INVENTION

1. Field of the Invention

The present invention relates to a process for manufacturing magnesium alkoxide and its use.

2. Discussion of the Background

Magnesium alkoxide can be used in the manufacture of ether. Here, a magnesium alkoxide is converted with an alkyl halide. In particular, magnesium alkoxides are used with such ether syntheses, where otherwise hydrogen chloride is separated off from the alkyl halides, using other alkoxides such as alkali alkoxides, thus forming olefins as by-products. A dialkyl sulfate can also be converted with a magnesium alcoholate to form an ether.

Manufacturing magnesium alcoholates from an anhydrous alcohol and magnesium in the presence of mercury (II) chloride or iodine as catalyst is known [Chem. Abstr. 66, 37426 g (1967)]. Magnesium is mostly used in a finely distributed form-powder or chips or beads. However, with some alcohols, such as methanol, after a delayed start to the reaction, the reaction proceeds very vigorously and can be uncontrolled. This can represent a problem for manufacturing magnesium methanolate from methanol and magnesium, in particular on an industrial scale.

The object was therefore to make available a process, wherein the magnesium reacts with methanol to completion in a controlled manner. A special objective of the present invention was to manufacture magnesium methanolate in methanolic solution or in solid form with a high quality and good yield.

This task is solved according to the present invention corresponding to the details of the patent claims.

### SUMMARY OF THE INVENTION

A process for manufacturing magnesium alkoxide was accordingly discovered, wherein, surprisingly, magnesium in piece form, e.g. bars, was converted in a reaction apparatus with excess alcohol, into magnesium alkoxide in a controlled process in a particularly simple and economic manner, wherein the reaction of the magnesium used was almost fully completed.

The object of the present invention is therefore a process for manufacturing magnesium alkoxide by converting magnesium with an alcohol characterized in that magnesium in piece form is used.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 exemplifies the configuration of an apparatus for practicing the reaction of magnesium with an alcohol.

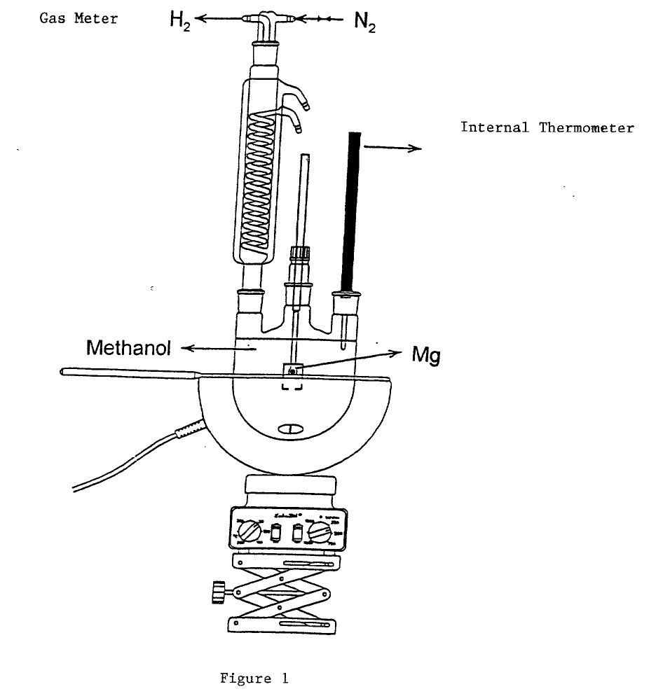

### DETAILED DESCRIPTION OF THE PREFERRED EMBODIMENTS

Appropriately, the magnesium pieces used in the process according to the present invention have on average at least one geometric, that is, external surface area of >10 cm², appropriately from 20 to 20,000 cm², preferably from 40 to 1,000 cm², in particular preferably from 50 to 500 cm², and most particularly preferably from 70 to 300 cm².

In a preferred embodiment, the magnesium pieces have a surface area to volume ratio of from 1 to 7 cm⁻¹, preferably 2 to 6 cm⁻¹, more preferably from 3 to 5 cm⁻¹.

The magnesium used in the process according to the present invention preferably has a block form such as bars, pigs, balls or beads, or a columnar form such as magnesium rods and bars, or a plate shape, wherein the minimum thickness or the minimum diameter of the magnesium pieces is preferably >1.0 cm.

In general, the aim is to use the purest possible magnesium. The magnesium used in the process according to the present invention appropriately has a magnesium content of >99% by weight.

Pieces of metallic magnesium used in the present process can also be freed of clinging impurities prior to conversion, for example, an oxide, hydroxide or carbonate layer can be removed by etching or polishing or grinding or sawing, to mention just a few possibilities.

With the process according to the present invention, methanol is used, in general, in excess, wherein the methanol functions as an adduct constituent of the reaction medium and as a solvent at the same time. Dry methanol is preferably used.

With the process according to the present invention, conversion of the magnesium pieces with methanol is performed preferably at a temperature in the range of 0 to 200° C., in particular preferably at 15 to 70° C., and most particularly preferably at 50 to 65° C. The conversion according to the present invention is appropriately carried out at a pressure in the range of 0.1 to 100 bar abs. Conversion is preferably carried out at 1 to 20 bar abs., and most particularly preferably at 1 to 5 bar abs.

The process of the present invention may be practice with any alcohol, but in particular is useful for the preparation of magnesium methanolate, and magnesium ethanolate, by the reaction of magnesium with methanol and ethanol.

With the process according to the present invention, conversion occurs nearly quantitatively, wherein a methanolic solution of magnesium methanolate having a high degree of purity is obtained. If after a predetermined, that is, limited reaction period there should be metallic magnesium residue present during the present process, this can be separated by physical means such as filtration or centrifugation, for example.

The process according to the present invention is preferably operated in such a manner that liquid methanol and solid magnesium are used as adducts in a mass ratio yielding a methanolic solution having a magnesium methanolate content of 0.1 to 30% by weight. Methanolic solutions are preferred which have a magnesium methanolate content of 1 to 15% by weight, in particular 3 to 10% by weight.

Solid magnesium methanolate can be obtained by evaporation of the solution resulting from the process according to the present invention.

By way of example, conversion of the educts methanol and magnesium can be performed according to the present invention in a reaction apparatus, as seen in diagram 1.

The process according to the present invention is generally performed as follows:

The reaction is believed to run in accordance with reaction equation 1:

Mg+2 CH₃OH→Mg(OCH₃)₂+H₂ (1)

The following reactions may arise as secondary reactions:

Mg(OCH₃)₂+2H₂O→Mg(OH)₂+2CH₃OH (2)

Mg(OCH₃)₂+CO₂+H₂O→MgCO₃+2CH₃OH (3)

The process according to the present invention should therefore be carried out with the exclusion of water and carbon dioxide. Measures for working without water and carbon dioxide are known in general, to those of ordinary skill in the art.

The reactor utilized for the present process, for example a laboratory three-necked flask, is appropriately equipped with temperature measurement equipment, such as an internal thermometer, gas volume measurement equipment, such as gas meter, a reflux condenser, inert gas blanketing, for example with dry nitrogen, a magnesium clamp, if required, heating, such as a heating mantle, and an agitator, such as a blade stirrer or magnetic stirrer with magnetic core (cf. diagram 1), which constantly provides for thorough intermixing or magnesium being flowed around by methanol during conversion.

For the purposes of carrying out the process the apparatus is usually first rinsed with a dry inert gas, such as nitrogen, and annealed at the same time. The magnesium can now be introduced into the reaction area of the unit, appropriately under inert gas. The magnesium can also be fixed in the reaction chamber to a suspension arrangement. Tap water, for example, is appropriate as a cooling medium for operating the reflux converter. The reactor can now be filled with preferably dried methanol in such a way that the magnesium is flowed around by methanol on all sides. The conversion according to the present invention is appropriately carried out under boiling conditions. Under normal conditions, the methanol boils at around 64° C. Conversion between methanol and pieces of magnesium can be carried out in a suitable reactor under reduced or increased pressure at a lower or higher temperature. After about 30 minutes, the reaction of the methanol with the magnesium generally sets in and the associated generation of hydrogen can be clearly read from the gas meter. Conversion is completed when all the magnesium has finished reacting and there is no further generation of hydrogen indicated on the gas meter. Conversion runs usually between 1 and 60 hours, preferably 12±4 hours, though the process can be terminated earlier. The result is generally a clear to slightly cloudy methanolic solution. It can additionally be filtered under inert gas. If a solid magnesium methanolate is aimed for as a product, the solution obtained according to the inventive process is concentrated. The pressure can be lowered from ambient pressure, 1 bar abs., to <1 mbar abs. for this purpose. At the same time, the temperature can be raised from 64° C. to 150° C. for example in the interior of the flask. In general product yields of >95% (relative to the magnesium used) are achieved with the process according to the present invention. In particular, yields of 90 to 100% are achieved. The purity of the products manufactured according to the present invention is generally >98%, with respect to magnesium methanolate, preferably 98 to 99.99%.

Magnesium methanolate obtained according to the inventive process can be utilized both as a methanolic solution and in solid form extremely well as feedstock for chemical and pharmaceutical processes, for manufacturing catalysts, preferably for polymerization catalysts, and as a neutralizing agent, in particular as an anhydrous neutralizing agent.

Having generally described this invention, a further understanding can be obtained by reference to certain specific examples which are provided herein for purposes of illustration only and are not intended to be limiting unless otherwise.

### EXAMPLES

### Example 1

29.7 g Mg in block form (dimensions: 1.5×1.5×7.5 cm) and 1317.8 g methanol are filled into a 2-liter apparatus as illustrated in diagram 1 and described in the text. The apparatus is rinsed with nitrogen, cooling water is fed into the reflux converter and the contents are heated to boiling point (boiling temperature ca. 64° C.). After a further approx. 30 minutes the reaction sets in and the generation of hydrogen can be read from the gas meter. The maximum hydrogen generation is 7 I/h. The reaction is concluded after some 12 hours. The result is 1291 g of magnesium methylate solution having a Mg content of 2.2% by weight. The total of Mg(OH)₂ and MgCO₃ is 0.04% by weight. Yield is 96%.

### Example 2

The solution obtained according to Example 1 is transferred to a Rotavapor where it is concentrated by evaporation under reduced pressure. Drying is completed at a temperature of around 150° C. and a pressure of <1 mbar. The result is a white powder (97.6 g corresponding to a yield of 97%) having a Mg value of 27.8%.

### Comparative Example

22 g Mg shavings and 1000 g methanol are placed in a 2-liter three-necked flask equipped with reflux condenser, internal thermometer and agitator. The reaction sets in by itself, lasting about 30 minutes. Thereafter, a very vigorous reaction (sharp rise in temperature) commences which can be managed only by the use of dry ice cooling. After a further 25 minutes the reaction levels off and the magnesium finishes reacting fully. Total duration of reaction is around 2.5 hours.

Obviously, numerous modifications and variations of the present invention are possible in light of the above teachings. It is therefore to be understood that within the scope of the appended claims, the invention may be practiced otherwise than as specifically described herein.

This application is based on German patent application 10045356.2 filed in the German Patent Office on Sep. 14, 2000, the entire contents of which are hereby incorporated by reference.

1. A process for manufacturing magnesium alkoxide comprising:
reacting magnesium with an alcohol,
wherein said magnesium is used in piece form.

2. The process of claim 1, wherein said magnesium has a geometric surface area of at least 10 cm².

3. The process of claim 1, wherein a single magnesium piece is used having a geometric surface of at least 10 cm².

4. The process of claim 1, wherein the magnesium used has a shape selected from the group consisting of a block, a column, a plate form or a mixture thereof.

5. The process of claim 1, wherein conversion is carried out at a temperature in the range of 0 to 200° C.

6. The process of claim 1, wherein conversion is carried out at 0.1 to 100 bar abs.

7. The process of claim 1, wherein said alcohol is methanol.

8. The process of claim 7, wherein liquid methanol and solid magnesium are used as adducts in a mass ratio yielding a methanolic solution having a magnesium methanolate content of 0.1 to 30% by weight.

9. The process of claim 7, wherein solid magnesium methanolate is obtained by evaporation of the resulting solution.

10. In a process for producing a chemical or pharmaceutical with a magnesium alkoxide, the improvement comprising preparing said the magnesium alkoxide according to the process of claim 1.

11. In a process for producing a catalyst from a magnesium alkoxide, the improvement comprising preparing said the magnesium alkoxide according to the process of claim 1.

12. In a process for producing a neutralizing agent with a magnesium alkoxide, the improvement comprising preparing said the magnesium alkoxide according to the process of claim 1.

13. The process of claim 10, wherein said magnesium alkoxide is magnesium methanolate.

14. The process of claim 11, wherein said magnesium alkoxide is magnesium methanolate.

15. The process of claim 12, wherein said magnesium alkoxide is magnesium methanolate.

16. The process of claim 1, wherein said magnesium pieces have a surface area to volume ratio from 1 to 7 cm⁻¹.
